(19) United States
(12) Patent Application Publication
(10) Pub. No.: US 2019/0328963 A1
(43) Pub. Date: Oct. 31, 2019

(54) INFUSION DEVICE SUITABLE TO TEST FOR EXTRAVASATION

(71) Applicant: Fresenius Vial SAS, Brézins (FR)

(72) Inventors: Rémy Wolff, Morette (FR); Christine Verne, Lyon (FR); Christine Durand, Chassieu (FR)

(21) Appl. No.: 16/461,363

(22) PCT Filed: Nov. 17, 2017
(86) PCT No.: PCT/EP2017/079561

§ 371 Date: May 15, 2019

(30) Foreign Application Priority Data
Dec. 9, 2016 (EP) 16306652.5

(51) Int. Cl. A61M 5/168; A61M 5/145

(52) U.S. Cl. CPC A61M 5/16859; A61M 2005/1588; A61M 5/1452

(57) ABSTRACT

An infusion device (1) for administering a medical fluid to a patient (P) comprises a pumping mechanism (11) for pumping a medical fluid through a delivery line (3), and a processor device (15) for controlling the pumping mechanism (11), during an infusion operation, to pump the medical fluid through the delivery line (3) in a downstream direction (F1). Herein, the processor device (15) is constituted to perform, prior to an infusion operation, a test function during which the processor device (15) controls the pumping mechanism (11) to pump the medical fluid through the delivery line (3) in an upstream direction (F2) opposite the downstream direction (F1). In this way an infusion device is provided that supports a user, in particular a nurse, to perform an infusion operation, in particular to set up the infusion device for performing the infusion operation.

The invention relates to an infusion device for administering a medical fluid to a patient according to the preamble of claim 1 and to a method for operating an infusion device.

An infusion device of this kind comprises a pumping mechanism for pumping a medical fluid through a delivery line, and a processor device for controlling the pumping mechanism, during an infusion operation, to pump the medical fluid through the delivery line in a downstream direction.

An infusion device of this kind may be a syringe pump or a volumetric (peristaltic) infusion pump.

Within a syringe pump, a medical fluid (such as a medication or a nutritional fluid for example for the parenteral feeding of a patient) is contained in a cylindrical tube of a syringe. By continuously pushing a piston of the syringe into the cylindrical tube the medical fluid is delivered out of the cylindrical tube through a suitable delivery line towards a patient for infusing the medical fluid into the patient.

In a volumetric (peristaltic) infusion pump, in contrast, a pumping mechanism acts, in a peristaltic fashion, onto a delivery line, for example onto a pump segment of the delivery line comprising a flexible membrane, such that fluid is pumped through the delivery line towards a patient.

Within a syringe pump, a force sensor is for example placed on a driving element (a so called pusher device) of the pumping mechanism of the infusion device acting onto the piston of the syringe. By measuring the force exerted on the piston the pressure within the cylindrical tube of the syringe (which is connected to the delivery line) can be derived.

In a volumetric pump, in contrast, a pressure may be measured for example directly on the delivery line using a suitable force sensor, which is placed on the pump such that it is in contact with the delivery line.

Generally, the pressure within the delivery line during a normal infusion process can be assumed to be (almost) 0, due to the resistance for delivering the medical fluid towards the patient Bing small. However, if an occlusion is present in the delivery line downstream of the pumping device, the pressure in the delivery line will rise above zero, which can be detected via the force sensor and which can be used to trigger an alarm if the pressure exceeds a certain preset threshold value.

One hazard linked to an intravenous infusion is extravasation. Extravasation occurs if, inadvertently, during an intravenous infusion a vesicant solution or medication leaks, from a desired infusion route, into the surrounding tissue. Tissue damage due to extravasation generally does not occur frequently, but, if it occurs, the consequences may be severe. The likelihood of an extravasation injury may for example depend on the age, the state of consciousness and the venous circulation of a patient and also of the type, location and placement of the intravenous catheter. Extravasation injury is induced most frequently by drugs that have high osmolalities, vesicant properties, or the ability to induce ischemia.

If a nurse prior to performing an infusion operation places a catheter intravenously within a patient, the nurse typically by hand aspirates blood from the vein using a syringe. If the catheter is correctly placed in the patient, blood will be aspirated easily. Else, in case the catheter has not been placed correctly, blood may hardly be aspirated.

Generally, when placing a catheter in a patient for performing an infusion operation, the placement of the catheter on the patient should be checked by a nurse. If correct placement of the catheter is ensured, the likelihood for extravasation is substantially reduced. Current practice however leaves it up to the nurse and his/her training and experience to test for a correct placement of the catheter, giving rise to some uncertainty.

It is an object of the invention to provide an infusion device and a method for operating an infusion device that support a user, in particular a nurse, to perform an infusion operation, in particular to set up the infusion device for performing the infusion operation.

This object is achieved by means of an infusion device comprising the features of claim 1.

Accordingly, the processor device is constituted to perform, prior to an infusion operation, a test function during which the processor device controls the pumping mechanism to pump the medical fluid through the delivery line in an upstream direction opposite the downstream direction.

Hence, a test function is proposed which allows a user, in particular a nurse, to use the infusion device to check for correct placement of a catheter on a patient prior to performing an infusion operation. In particular, the infusion device is constituted and configured to automatically perform a reverse pumping action by pumping fluid in the upstream direction in order to aspirate body fluid (blood in case a catheter connected to the delivery line is placed intravenously in the patient). A task that conventionally had to be done by hand by a user, hence, now is taken over by the infusion device, which offers the advantage that the test function is performed in a reproducible manner allowing to check for a correct placement of a catheter according to predefined criteria.

By checking the correct placement of a catheter in a patient the likelihood for extravasation can substantially be reduced. In particular, if it is ensured that the catheter is placed correctly in a patient and hence an infusion enters into the infusion route that is desired, the likelihood that a medical fluid leaks from the infusion route into surrounding tissue at the location of the catheter is largely reduced.

In one embodiment, the infusion device comprises a sensor device for measuring a measurement value indicative of a pressure in the delivery line, wherein the processor device is constituted to evaluate, during the test function, measurement values obtained from the sensor device to identify a change in pressure in the delivery line during the test function. The infusion device is constituted to measure the (positive) force during a regular infusion operation while pushing on the piston of the syringe and the (negative) force during a reverse pumping action while pulling on the piston.

It is to be noted that the test function in principle works also without a sensor by prompting a user, for example a nurse, to check after the reverse pumping during the test function to check whether blood has been aspirated or not.

The test function may for example be triggered by a user by inputting a corresponding input command into an input device of the infusion device. The infusion device for example may comprise a dedicated button which a user may press to initiate the performing of the test function. The infusion device herein, when setting up the infusion operation, may propose to a user to initiate the test function, wherein the infusion device may further be constituted to allow the start of a subsequent infusion operation if and only if the test function has successfully been performed.

In one embodiment, the infusion device may be constituted to output information relating to a change in pressure during the test function. Generally, during the reverse pumping when performing the test function the pressure in the delivery line will decrease and become a negative, wherein the resulting pressure curve will largely depend on the connection of the delivery line to the patient. If a catheter connected to the delivery line is incorrectly placed on a patient, fluid will be drawn into the delivery line with a much larger resistance than in case of a correct placement of the catheter. Hence, if the catheter is placed incorrectly in the patient, the change in pressure during the test function will be much greater than in case of a correct placement of the catheter, the reverse pumping causing a negative pressure within the infusion line having a relatively large magnitude. Hence, from the absolute change in pressure and/or from the slope of the pressure during the test function it can be derived whether the catheter is placed correctly on the patient or not.

The infusion device may for example be constituted to output absolute numbers of the change in pressure or the slope of the pressure curve on a display device such that a user, for example a nurse, may see from the change in pressure and/or from the slope of the pressure curve whether the catheter is placed correctly or incorrectly on the patient. In addition or alternatively, the infusion device may output a warning message, for example a text message on a display device or a sound message via a suitable acoustic alarm system, the message warning a user that potentially the catheter is placed incorrectly on the patient.

In one embodiment, the control device is constituted to terminate the test function according to at least one criteria observed during the performing of the test function. Herein, different criteria may exist which may be observed and which may lead to a termination of the test function.

For example, the control device may be constituted to perform the test function for a predetermined amount of time. Hence, the pumping mechanism is controlled to reversely pump fluid for a predetermined amount of time, and upon lapse of the predetermined time the test function is terminated.

In addition or alternatively, the control device may be constituted to terminate the test function in case the pressure in the delivery line falls under a predetermined negative pressure threshold. The pressure in the delivery line is derived from sensor readings of the sensor device. During the test function the pressure in the delivery line will drop due to the reverse pumping action of fluid in the upstream direction. If it is found that the pressure drops under a predetermined negative threshold (i.e. the magnitude of the negative pressure exceeds the magnitude of the threshold), it is concluded that potentially an abnormal condition is present, possibly due to an incorrect placement of the catheter connected to the delivery line and the patient. Due to an incorrect placement of a catheter fluid may experience a fairly large resistance when drawn into the delivery line, leading to a substantial drop of the pressure in the delivery line (a rise of the negative pressure). This can be observed and, by comparison to a predefined threshold, be used as a termination criteria.

Alternatively or in addition, the volume of fluid pumped in the upstream direction during the test function may be observed. If it is found that the volume pumped during the test function exceeds a predetermined maximum volume, the control device may terminate the test function. Hence, it is ensured that not more than a maximum volume of fluid is pumped in the reverse direction during the test function.

Alternatively or in addition, the control device may be constituted to terminate the test function in case a user releases an input device. For example, the infusion device may be configured such that the test function is carried out as long a user presses a dedicated button on the infusion device. If the user releases the button, the test function may terminate automatically.

In a further embodiment, the control device may be constituted to control the pumping mechanism, after termination of the test function, to pump fluid through the delivery line in the downstream direction to reinject fluid pumped in the upstream direction during the test function. Hence, after termination of the test function, the volume of fluid pumped in the upstream direction is reinjected into the patient such that the pumped volume is re-delivered into the patient. Beneficially, the reinjected volume herein matches (substantially exactly) the volume which has been pumped in the upstream direction during the test function, such that the overall net volume moved over the test function and its subsequent reinjection operation is zero.

The infusion device may be of different constitutions and shapes. For example, the infusion device may be a syringe pump in which a syringe is placed in a receptacle, a pusher device acting onto a piston of the syringe in order to deliver a medical fluid from a cylindrical tube of the syringe into a delivery line connected to the cylindrical tube. In another embodiment, the infusion device may be a volumetric (peristaltic) infusion pump, in which a pumping mechanism in a peristaltic fashion acts onto the delivery line in order to pump fluid through the delivery line. In yet another embodiment, the infusion device may have an entirely different constitution and shape. The invention is thus not limited to any particular kind of infusion device.

The object is also achieved by a method for operating an infusion device for administering a medical fluid to a patient. The method comprises:
pumping a medical fluid through a delivery line using a pumping mechanism, and
controlling, using a processor device, the pumping mechanism, during an infusion operation, to pump the medical fluid through the delivery line in a downstream direction, Herein, prior to an infusion operation a test function is performed during which the processor device controls the pumping mechanism to pump the medical fluid through the delivery line in an upstream direction opposite the downstream direction.

Herein, in one embodiment a measurement value indicative of a pressure in the delivery line using a sensor device is measured, wherein the processor device evaluates measurement values obtained from the sensor device to identify a change in pressure in the delivery line during the test function.

The advantages and advantageous embodiments described above for the infusion device equally apply also to the method such that it shall be referred to the above.

The idea of the invention shall subsequently be described in more detail with reference to the embodiments shown in the figures. Herein:

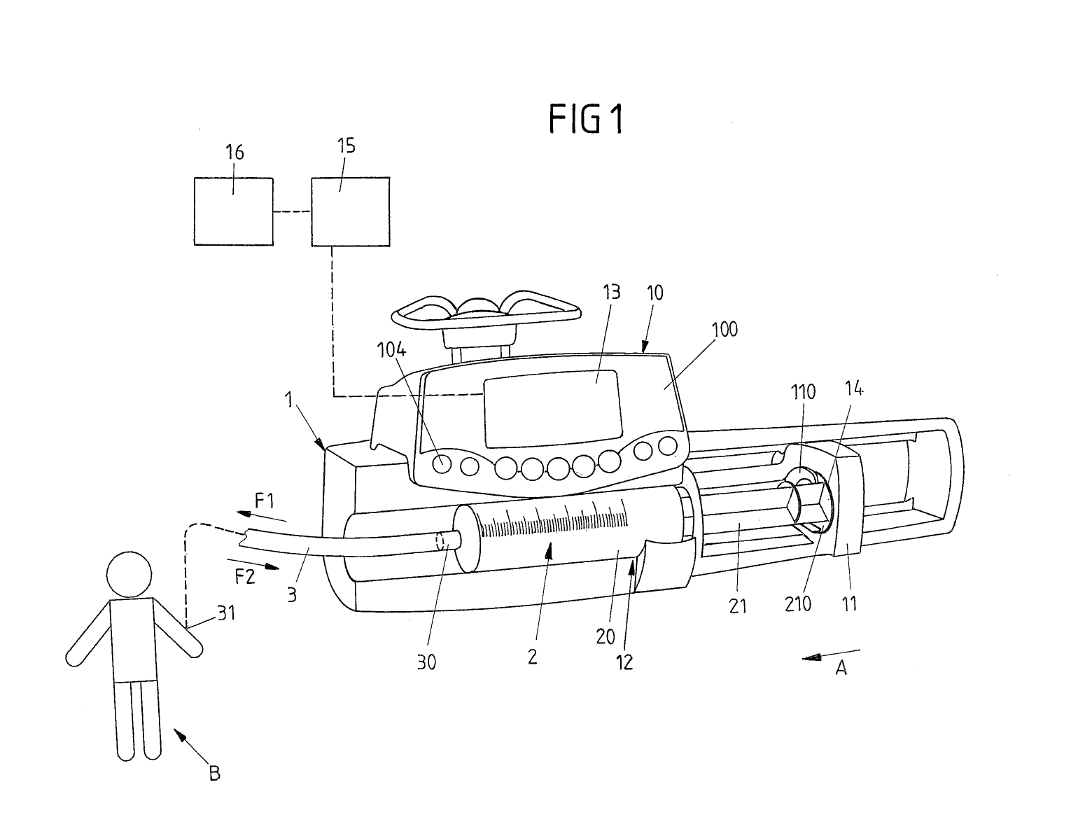

FIG. 1 shows a view of an infusion device constituted as a syringe pump;

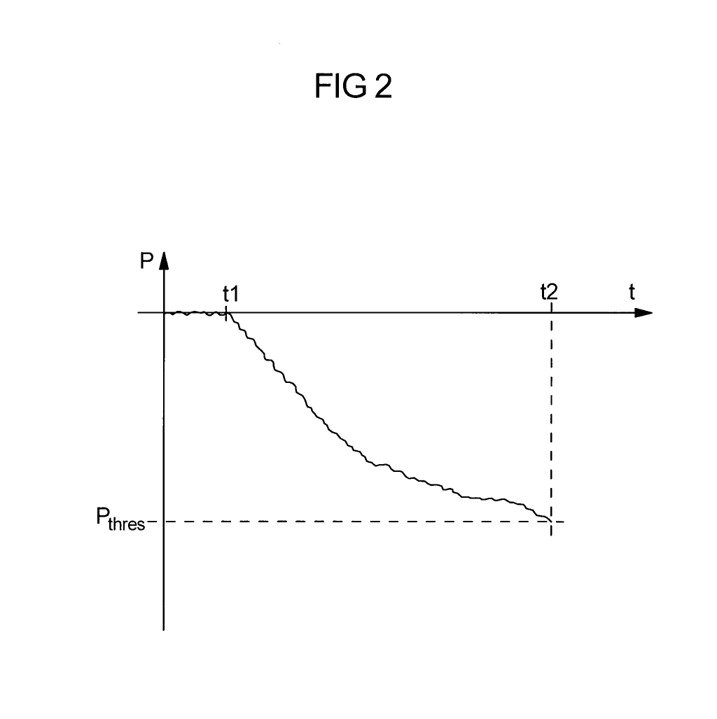

FIG. 2 shows a graphical view of the measured pressure during a reverse pumping operation; and

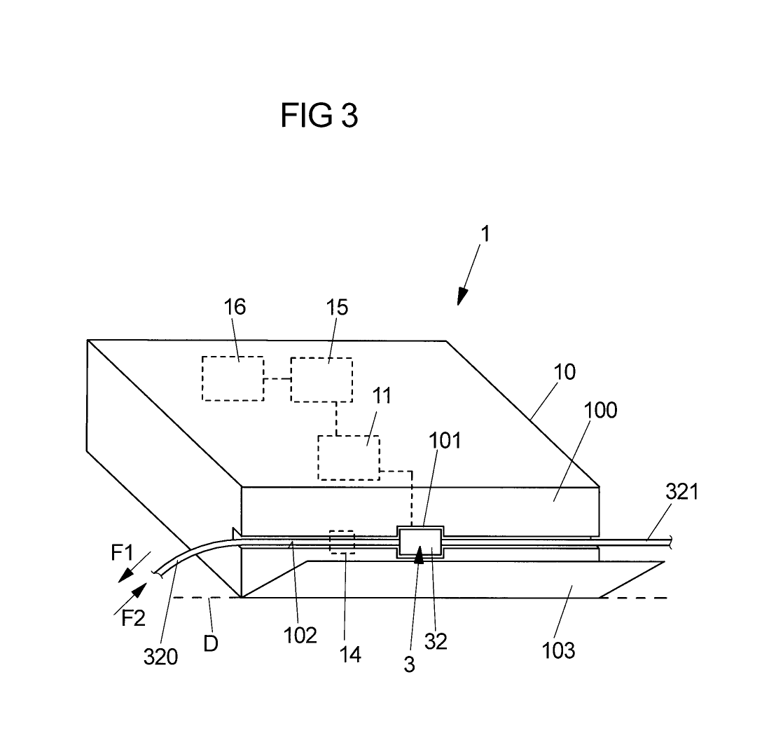

FIG. 3 shows a view of an infusion device constituted as a volumetric (peristaltic) infusion pump.

FIG. 1 shows an embodiment of an infusion device 1 in the shape of a syringe pump.

The infusion device 1 comprises a housing 10 having a front face 100 and a display device 13 arranged thereon. The display device 13 may for example be a touch-sensitive display allowing a user to enter commands for operation of the infusion device 1 and displaying operational information regarding the process of an actual infusion operation.

The infusion device 1 comprises a receptacle 12 in which a syringe 2 having a cylindrical tube 20 is arranged. A piston 21 is movable within the cylindrical tube 20 and is in engagement with a pusher device 11 of a pumping mechanism of the infusion device 1. At an end of the cylindrical tube 20 opposite the piston 21 a delivery line 3 extends from the cylindrical tube 20 towards a patient B, the delivery line 3 being connected to the cylindrical tube 20 at an end 30 and to the patient B at an end 31.

The piston 21 comprises a head 210 facing away from the cylindrical tube 20 and being in abutment with the pusher device 11 of the infusion device 1. The piston head 210, when the syringe 2 is placed in the receptacle 12, is fixed to the pusher device 11 by means of a clamping device 110 reaching around the piston head 210, such that the piston head 210 is held in tight abutment with the pusher device 11. During operation of the infusion device 1, the pusher device 11 is electromotorically driven in an actuation direction A such that the piston 21 is moved into the cylindrical tube 20 and a medical fluid contained in the cylindrical tube 20 is delivered via the delivery line 3 towards the patient B.

The infusion device 1 comprises a processor device 15 and a storage device 16. Via the processor device 15 the infusion operation of the infusion device 1 is controlled. In the storage device 16 operational parameters, such as mechanical characteristics of the syringe 2 used on the infusion device 1 as well as operational data, may be stored.

During an infusion process a medical fluid, for example a medication or a nutritional fluid for the parenteral feeding of a patient or the like, is delivered from the cylindrical tube 20 via the delivery line 3 towards the patient B. For this, the piston 21 is continuously pushed into the cylindrical tube 20 in the actuation direction A such that a desired flow rate is obtained, which is programmed by a user prior to the start of the infusion operation.

The delivery line 3 generally is made of a flexible tubing made for example from a PVC material. The delivery line extends from the cylindrical tube 20 towards the patient B and is, at its first end 30, in fluid connection with the cylindrical tube 20 and, at its second end 31, for example connected to a catheter for providing an intravenous access to the patient B.

In between the pusher device 11 and the piston head 210 a force sensor 14 is placed which measures the force exerted on the piston head 210. The force sensor 14 is capable to measure a force when actuating the pusher device 11 to push the piston 21 in the actuation direction A into the cylindrical tube 20 in order to deliver a medical fluid from the cylindrical tube 20 in a downstream direction F1 towards the patient B. Likewise, due to the tight abutment in which the piston head 210 is held on the pusher device 11 by means of the clamping device 110, the force sensor 14 is capable also to measure the force between the pusher device 11 and the piston head 210 in case the pusher device 11 is actuated in a direction opposite to the actuation direction A such that the piston 21 is pulled out of the cylindrical tube 20 and a fluid is drawn in an upstream direction F2 through the delivery line 3.

The pusher device 11 and the force sensor 14 arranged thereon may for example be constituted as described in WO 2012/123417 A1, whose contents shall be incorporated by reference herein.

It is to be noted in particular that the infusion device 1 is constituted to measure the (positive) force during a regular infusion operation while pushing on the piston 21 of the syringe 2 and the (negative) force during a reverse pumping action while pulling on the piston 21.

To observe the pressure in the delivery line 3, the force applied to the piston head 210 of the piston 21 by means of the pusher device 11 is measured by the sensor 14 placed in between the pusher device 11 and the piston head 210 and abutting the piston head 210 for example with an elastic pretension. The force measured in this way allows for an indirect measurement of the pressure within the cylindrical tube 20, which generally equals the pressure in the delivery line 3.

The pressure in the cylindrical tube 20 during an infusion operation (during which the pusher device 11 pushes the piston 21 in the actuation direction A into the cylindrical tube 20) depends on the measured force according to the following relation:

$$P = \frac{F - {F}_{0}}{S} .$$

Herein, P denotes the pressure, F denotes the measured force, F₀ denotes a frictional force component and S denotes the effective surface by which the piston 21 acts onto the fluid contained in the cylindrical tube 20. The effective surface S is substantially determined by the inner diameter of the cylindrical tube 20. Whereas F is measured and S is known from the geometrical dimensions of the cylindrical tube 20 of the syringe 2, the frictional force component F₀ may be assumed as constant or may be determined for a particular syringe 2 using a calibration or a modeling. F₀ may dependent on the particular syringe 2 used and on the position of the piston 21 within the cylindrical tube 20.

During a reverse pumping, i.e. during a pulling of the piston 21 out of the cylindrical tube 20 by actuation of the pusher device 11 in a direction opposite to the actuation direction A, the pressure in the delivery line 3 can be deduced from the sensor readings of the force sensor 14 using the equation:

$$P = \frac{F + {F}_{0}}{S}$$

Hence, taking into account that the frictional force F₀ opposes the movement and hence changes signs, for deriving the pressure P in the delivery line 3 during the reverse pumping in the upstream direction F2 the frictional force component F₀ is used with opposite sign as compared to the equation noted above for the for the pumping in the forward, downstream pumping direction F1. Again, F is the force value measured by the force sensor 14, and S denotes the effective surface by which the piston 21 of the syringe 2 acts onto fluid contained in the cylindrical tube 20 of the syringe 2, determined by the inner parameter of the cylindrical tube 20. During reverse pumping, the measured force F can be expected to be negative, due to a pulling force exerted on the piston 21. If the measured force F during the reverse pumping is smaller than—F0, the (estimated) pressure P in the infusion line becomes negative.

Prior to starting an infusion operation, it must be made sure that a catheter linked to the delivery line 3 is correctly placed in the patient B and accesses the infusion route desired. In particular, if an intravenous infusion shall be performed, it must be made sure that the catheter is correctly placed in the vein that shall be accessed. If the catheter is not placed correctly in the patient B (because the catheter for example has missed the vein or has punctured the vein's wall) a potential risk of extravasation exists, i.e. a leaking of a (e.g.) vesicant fluid or medication into the surrounding tissue, which may bear the risk of severe injury for the patient B.

To check for the correct placement of the catheter, the processor device 15 of the infusion device 1 is constituted to perform a test function prior to the start of an infusion operation. The infusion device 1, in the embodiment shown in FIG. 1, comprises a number of dedicated buttons 104, one of which may be dedicated to the performing of the test function. If a user presses the button 104 associated with the test function, the infusion device 1 may automatically start and perform the test function. Herein, in one embodiment it may be provided that the test function is carried (at maximum) as long as a user presses the button 104. Hence, the user may be required to keep pressing the button 104 during performance of the test function, a release of the button 104 leading to a termination of the test function.

During the test function, the processor device 15 controls the pusher device 11 to actuate the piston 21 in a direction opposite to the actuation direction A. Hence, fluid is pumped in the upstream direction F2 such that body fluid is aspirated from the patient B. During the reverse pumping, the force on the piston 21 is measured by means of the force sensor 14 and the pressure in the delivery line 3 is monitored by the processor device 15.

Generally, if the catheter is placed correctly in the patient B and a reverse pumping is initiated, body fluid will be drawn out of the patient B at low resistance. In case the catheter is (correctly) placed in a vein of the patient B for an intravenous infusion, blood is aspirated. In case of a correct placement of the catheter, the pressure in the delivery line 3 will only slightly become negative (i.e., a small negative pressure will develop in the delivery line 3 due to the sucking force of the piston 21).

In case of an incorrect placement, in contrast, the resistance for drawing body fluid from the patient B is substantially increased, leading to a substantial drop in the pressure in the delivery line 3. Hence, a substantial negative pressure in the delivery line 3 will develop, which can be evaluated by the processor device 15 and which for example can be displayed on the display device 13 such that a user is noticed of the abnormal pressure drop in the delivery line 3.

For example, the processor device 15 may display a curve of the pressure on the display device 13. Alternatively or in addition, information relating to the absolute pressure drop and/or a slope of the pressure drop may be displayed on the display device 13.

The test function, during which fluid is reversely pumped in the upstream direction F2 by moving the pusher device 11 and (together with the pusher device 11) the piston 21 opposite to the actuation direction A, may be performed for a predetermined amount of time or for a predetermined volume. In the latter case, if it for example is found that the volume pumped in the upstream direction F2 is equal to or exceeds a predefined maximum value, the test function may be stopped.

In addition or alternatively, as illustrated in FIG. 2, the (negative) pressure in the delivery line 3 may be compared to a negative pressure threshold Pₜₕᵣₑₛ. If, in the example of FIG. 2, the test function is initiated at time t1 and the pressure in the infusion line 3 becomes smaller than the negative pressure threshold Pₜₕᵣₑₛ at the time t2, the test function may be terminated at the time t2 and a corresponding warning message may be displayed on the display device 13.

When an abnormal condition during the test function arises, for example due to the pressure in the infusion line 3 dropping below the negative pressure threshold Pₜₕᵣₑₛ, a warning message can be displayed on the display device 13 comprising relevant information about the pressure drop and/or a text message warning about a potential wrong placement of the catheter, wherein alternatively or additionally an acoustic alarm message may be triggered.

After termination of the test function, the processor device 15, in one embodiment, controls the pusher device 11 to pump a volume of fluid in the downstream direction F1 to reinject the volume of fluid that has been reversely pumped during the test function. Hence, the body fluid aspirated during the test function is redelivered to the patient B. Beneficially, the volume of fluid pumped in the upstream direction F2 during the test function matches the volume of fluid which is reinjected after termination of the test function in the downstream direction F1, such that the net sum of the pumped fluid is zero.

The invention is not limited to syringe pumps as shown in FIG. 1. Rather, as illustrated in FIG. 3, the invention likewise is applicable also to volumetric pumps.

In the example of FIG. 3, an infusion device 1 is constituted as a volumetric (peristaltic) infusion pump having a housing 10 and a front face 100 comprising a receptacle 101 in which a pump module 32 of an infusion set 3 can be received. A housing element 103 (also denoted as the “door”) is pivotable about a pivoting axis D relative to the housing 10 and can be approached towards the front face 100 such that in a closed state the tubing set 3 is held on the infusion device 1.

From the pump module 32 tube sections 320, 321 extend. Of these tube sections 320, 321, an upstream tube section 321 connects the pump module 32 to a container containing a medical fluid, whereas a downstream pump section 320 connects the pump module 32 to a patient B for delivering the medical fluid towards the patient B. The infusion device 1 comprises a pumping mechanism 11 (illustrated only schematically in FIG. 3) having for example a wobbling device acting onto the pump module 32 for peristaltically pumping a medical fluid through the tubing set 3 towards the patient B.

The invention may be implemented in the infusion device 1 according to FIG. 3 in an analogous fashion as described above, such that it shall be referred to the above. Herein, the pumping mechanism 11 of the infusion device 1 is constituted to pump fluid in the downstream direction F1 during a regular infusion operation and in the upstream direction F2 when performing the test function.

The idea of the instant invention is not limited to the embodiments described above, but may be carried out in an entirely different way in entirely different embodiments.

### LIST OF REFERENCE NUMERALS

1 Infusion device
10 Housing
100 Front face
101 Receptacle
102 Channel
103 Housing element (door)
104 Input device
11 Pumping mechanism (pusher device)
110 Clamping device
12 Receptacle
13 Display device
14 Force sensor
15 Processor device
16 Storage device
2 Pumping device (syringe)
20 Cylindrical tube
21 Piston
210 Piston head
3 Delivery line
30, 31 End
32 Pump module
320, 321 Tube section
A Actuation direction
B Patient
D Pivoting axis
F1 Downstream direction
F2 Upstream direction
P Pressure
Pₜₕᵣₑₛ Pressure threshold
t time

1. An infusion device for administering a medical fluid to a patient, comprising:
a pumping mechanism for pumping a medical fluid through a delivery line, and
a processor device for controlling the pumping mechanism, during an infusion operation, to pump the medical fluid through the delivery line in a downstream direction,
wherein the processor device is configured to perform, prior to an infusion operation, a test function during which the processor device controls the pumping mechanism to pump the medical fluid through the delivery line in an upstream direction opposite the downstream direction.

2. The infusion device according to claim 1, further comprising a sensor device for measuring a measurement value indicative of a pressure in the delivery line, wherein the processor device is configured to evaluate, during the test function, measurement values obtained from the sensor device to identify a change in pressure in the delivery line during the test function.

3. The infusion device according to claim 1, further comprising an input device enabling a user to input a control command to trigger the processor device to perform the test function.

4. The infusion device according to claim 1, further comprising an output device for outputting information relating to a change in pressure during the test function.

5. The infusion device according to claim 1, wherein the control device is configured to terminate the test function according to at least one criteria observed during the performing of the test function.

6. The infusion device according to claim 1, wherein the control device is configured to terminate the test function in case the pressure in the delivery line falls under a predetermined negative pressure threshold.

7. The infusion device according to claim 1, wherein the control device is configured to terminate the test function in case the volume of fluid pumped in the upstream direction during the test function exceeds a predetermined maximum volume.

8. The infusion device according to claim 1, wherein the control device is configured to terminate the test function in case an input device is released.

9. The infusion device according to claim 1, wherein the control device is configured to control the pumping mechanism, after termination of the test function, to pump fluid through the delivery line in the downstream direction to reinject fluid pumped in the upstream direction during the test function.

10. The infusion device according to claim 9, wherein the control device is configured to control the pumping mechanism to reinject a volume of fluid matching the volume of fluid pumped in the upstream direction during the test function.

11. The infusion device according to claim 1, wherein the infusion device is a syringe infusion pump or volumetric infusion pump.

12. A method for operating an infusion device for administering a medical fluid to a patient, comprising:
pumping a medical fluid through a delivery line using a pumping mechanism,
controlling, using a processor device, the pumping mechanism, during an infusion operation, to pump the medical fluid through the delivery line in a downstream direction,
and
performing, prior to an infusion operation, a test function during which the processor device controls the pumping mechanism to pump the medical fluid through the delivery line in an upstream direction opposite the downstream direction.

13. The method according to claim 12, further comprising measuring a measurement value indicative of a pressure in the delivery line using a sensor device, wherein the processor device evaluates measurement values obtained from the sensor device to identify a change in pressure in the delivery line during the test function.
